(19) United States
(12) Patent Application Publication
(10) Pub. No.: US 2011/0124729 A1
(43) Pub. Date: May 26, 2011

(54) USE OF HIGHLY-BRANCHED POLYESTERS IN COSMETIC AND DERMATOLOGICAL FORMULATIONS

(75) Inventors: Volker Wendel, Seeheim-Jugenheim (DE); Matthias Laubender, Schifferstadt (DE); Jean-Fracois Stumbe, Strasbourg (FR); Bernd Bruchmann, Freinsheim (DE)

(73) Assignee: BASF SE, Ludwigshafen (DE)

(21) Appl. No.: 12/952,711

(22) Filed: Nov. 23, 2010

Related U.S. Application Data
(60) Provisional application No. 61/264,675, filed on Nov. 26, 2009.

(51) Int. Cl. A61K 8/85; A61Q 19/00; C07C 69/34

(52) U.S. Cl. 514/547; 560/190

(57) ABSTRACT

The present invention relates to compositions which comprise highly branched polyesters, to the use of these highly branched polyesters in cosmetics and dermatology and to substituted highly branched polyesters.

The present invention relates to compositions which comprise highly-branched polyesters, and to the use of these highly-branched polyesters in cosmetics and dermatology.

Thickeners are used to a great degree in the field of pharmacy and cosmetics for increasing the viscosity of preparations.

The thickeners are chosen according to whether the preparation is aqueous, oily or surface-active. An overview on this topic is given in Hugo Janistyn, Handbuch der Kosmetika and Riechstoffe [Handbook of cosmetics and fragrances], Hüthig Verlag Heidelberg, volume 1, 3rd edition, 1978, p. 979.

Examples of thickeners that are often used for aqueous solutions are fatty acid polyethylene glycol monoesters, fatty acid polyethylene glycol diesters, fatty acid alkanolamides, oxyethylated fatty alcohols, ethoxylated glycerol fatty acid esters, cellulose ethers, sodium alginate, polyacrylic acids, and neutral salts.

Polymers comprising carboxyl groups are also known as thickeners. These include homopolymers and copolymers of monoethylenically unsaturated carboxylic acids, such as acrylic acid, methacrylic acid, maleic acid, maleic anhydride and itaconic acid. These polymers are often crosslinked at least to a small extent. Such polymers are described, for example, in U.S. Pat. No. 2,798,053, U.S. Pat. No. 3,915,921, U.S. Pat. No. 3,940,351, U.S. Pat. No. 4,062,817, U.S. Pat. No. 4,066,583, U.S. Pat. No. 4,267,103, U.S. Pat. No. 5,349,030 and U.S. Pat. No. 5,373,044.

Frequent disadvantages of these polymers when used as thickeners are their pH dependency and hydrolytic instability. Furthermore, large amounts of the polymers are often required for achieving the desired thickening effect, and the stability of the preparations in the presence of electrolytes is low.

Naturally occurring materials such as casein, alginates, methylcellulose, hydroxyethylcellulose, hydroxypropylcellulose and carbomethoxycellulose are also used as thickeners. These have, inter alia, the disadvantage of sensitivity to microbiological factors and the addition of biocides is consequently required. Typical thickeners of oily preparations, also called oil thickeners below, are metal soaps, amorphous silicon dioxide, hydroxystearin, compounds of quaternary ammonium bases with bentonites, waxes and paraffins.

Surfactant solutions are thickened, for example, by fatty acid alkylolamides, amine oxides, cellulose derivates, polysaccharides and the aforementioned polymers comprising carboxyl groups.

High-functionality highly-branched polyesters and processes for their preparation are described, for example, in DE 101 63 163, DE 102 19 508, DE 102 40 817, DE 103 48 463, DE 10 2004 026904 and DE 10 2005 060783.

WO 2006/018063 describes compositions for hair cosmetics which comprise hydrophobically functionalized dendritic macromolecules. The dendritic macromolecules are composed either of polyester units (obtainable under the trade name Boltorn) or of polyamide units (obtainable under the trade name Hybrane).

DE 10 2005 063 096 describes cosmetic compositions which comprise 0.05 to 20% by weight of at least one hyperbranched polyester and/or polyester amide. The compositions reportedly have hair cleansing and/or hair care properties. The polyesters and/or polyester amides are not substituted.

WO 2004/078809 discloses highly-branched polymers and cosmetic compositions comprising these.

It was an object of the present invention to find rheology-modifying, in particular thickening, in particular oil-thickening, polymers which are highly suitable for cosmetic applications and have good application properties especially in the field of skin cosmetics. Besides the good thickening effect for a small use of material, these also include clarity in the case of gel applications, (co-)emulsifying and stabilizing effect for oil-insoluble and/or difficult-to-stabilize components, good incorporability into cosmetic preparations. For gels in particular, the highest possible transparency (clarity) of the preparations is desired. In order to ensure the broadest possible formulatability, it is desired that the thickeners are low-color and low-odor, ideally colorless and odorless. Moreover, for use in (skin) cosmetic and/or dermatological applications, it is necessary that no allergenic reactions are triggered.

The object is achieved by the substituted highly-branched polyesters described below.

Within the context of this invention, highly-branched polyesters are understood as meaning uncrosslinked macromolecules with hydroxyl groups and carboxyl groups which are both structurally and also molecularly nonuniform. They can firstly be composed starting from a central molecule analogously to dendrimers, but with nonuniform chain length of the branches. They may secondly also be linear in composition, with functional side groups, or else, as a combination of the two extremes, have linear and branched molecular moieties. For the definition of dendrimeric and hyperbranched polymers, see also P. J. Flory, J. Am. Chem. Soc. 1952, 74, 2718 and H. Frey et al., Chem. Eur. J. 2000, 6, No. 14, 2499.

In connection with the present invention, “highly-branched” is understood as meaning that the degree of branching (DB), i.e. the average number of dendritic linkages plus the average number of end groups per molecule, divided by the sum of the average number of dendritic linkages, the average number of linear linkages and the average number of end groups, multiplied by 100, is 10 to 99.9%, preferably 20 to 99%, particularly preferably 20-95%.

Besides the expression highly-branched, the expression hyperbranched is also known from the literature. Within the context of the present invention, the two expressions should be understood synonymously.

In connection with the present invention, “dendrimeric” is understood as meaning that the degree of branching is 99.9-100%. For the definition of the degree of branching, see H. Frey et al., Acta Polym. 1997, 48, 30.

Within the context of this document, uncrosslinked means that a degree of branching of less than 15% by weight, preferably of less than 10% by weight, determined via the insoluble fraction of the polymer, is present.

The insoluble fraction of the polymer was determined by extraction for 4 hours with the same solvent as is used for the gel permeation chromatography, i.e. tetrahydrofuran, dimethylacetamide or hexafluoroisopropanol, depending on in which solvent the polymer is more soluble, in a Soxhlet apparatus and, after drying the residue to constant weight, weighing the remaining residue.

The highly-branched polyesters are prepared as described below.

At least one aliphatic, cycloaliphatic, araliphatic or aromatic dicarboxylic acid (A₂) or derivatives thereof and, if appropriate, a dihydric, aliphatic, cycloaliphatic, araliphatic or aromatic alcohol (B₂), which has 2 OH groups, are reacted with

at least one x-hydric aliphatic, cycloaliphatic, araliphatic or aromatic alcohol (Cₓ), which has more than two OH groups and x is greater than 2, preferably between 3 and 8, particularly preferably between 3 and 6, very particularly preferably from 3 to 4 and in particular 3,
if appropriate in the presence of further functionalized building blocks E,
where the ratio of the reactive groups in the reaction mixture is chosen so that a molar ratio of OH groups to carboxyl groups or derivatives thereof of from 5:1 to 1:5, preferably from 4:1 to 1:4, particularly preferably from 3:1 to 1:3 and very particularly preferably from 2:1 to 1:2 is established.

The dicarboxylic acids (A₂) include, for example, aliphatic dicarboxylic acids, such as oxalic acid, malonic acid, succinic acid, glutaric acid, adipic acid, pimelic acid, suberic acid, azelaic acid, sebacic acid, undecane-α,ω-dicarboxylic acid, dodecane-α,ω-dicarboxylic acid, cis- and trans-cyclohexane-1,2-dicarboxylic acid, cis- and trans-cyclohexane-1,3-dicarboxylic acid, cis- and trans-cyclohexane-1,4-dicarboxylic acid, cis- and trans-cyclopentane-1,2-dicarboxylic acid, cis- and trans-cyclopentane-1,3-dicarboxylic acid. Furthermore, it is also possible to use aromatic dicarboxylic acids, such as, for example, phthalic acid, isophthalic or terephthalic acid. Unsaturated dicarboxylic acids, such as maleic acid or fumaric acid, can also be used.

The specified dicarboxylic acids may also be substituted by one or more radicals, selected from

C₁-C₁₀-alkyl groups, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, isoheptyl, n-octyl, 2-ethylhexyl, trimethylpentyl, n-nonyl or n-decyl,
C₃-C₁₂-cycloalkyl groups, for example cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl;
alkylene groups such as methylene or ethylidene or
C₆-C₁₄-aryl groups, such as, for example, phenyl, 1-naphthyl, 2-naphthyl, 1-anthryl, 2-anthryl, 9-anthryl, 1-phenanthryl, 2-phenanthryl, 3-phenanthryl, 4-phenanthryl and 9-phenanthryl, preferably phenyl, 1-naphthyl and 2-naphthyl, particularly preferably phenyl.

Examples of representatives of substituted dicarboxylic acids that may be mentioned are: 2-methylmalonic acid, 2-ethylmalonic acid, 2-phenylmalonic acid, 2-methylsuccinic acid, 2-ethylsuccinic acid, 2-phenylsuccinic acid, itaconic acid, 3,3-dimethylglutaric acid.

Furthermore, mixtures of two or more of the aforementioned dicarboxylic acids can be used.

The dicarboxylic acids can be used either as they are or in the form of derivatives.

Derivatives are preferably understood as meaning
the relevant anhydrides in monomeric or else polymeric form,
mono- or dialkyl esters, preferably mono- or di-C₁-C₄-alkyl ester, particularly preferably mono- or dimethyl esters or the corresponding mono- or diethyl esters,
also mono- and divinyl esters, and
mixed esters, preferably mixed esters with different C₁-C₄-alkyl components, particularly preferably mixed methylethyl esters.

Within the context of this document, C₁-C₄-alkyl is methyl, ethyl, isopropyl, n-propyl, n-butyl, isobutyl, sec-butyl and tert-butyl, preferably methyl, ethyl and n-butyl, particularly preferably methyl and ethyl and very particularly preferably methyl.

Within the context of the present invention, it is also possible to use a mixture of a dicarboxylic acid and one or more of its derivatives. Within the context of the present invention, it is likewise possible to use a mixture of two or more different derivatives of one or more dicarboxylic acids.

Particular preference is given to using malonic acid, succinic acid, glutaric acid, adipic acid, 1,2-, 1,3- or 1,4-cyclohexanedicarboxylic acid (hexahydrophthalic acids), phthalic acid, isophthalic acid, terephthalic acid or mono- or dialkyl esters thereof.

Diols (B₂) that can be used according to the present invention are, for example, ethylene glycol, propan-1,2-diol, propane-1,3-diol, butane-1,2-diol, butane-1,3-diol, butane-1,4-diol, butane-2,3-diol, pentane-1,2-diol, pentan-1,3-diol, pentane-1,4-diol, pentane-1,5-diol, pentane-2,3-diol, pentane-2,4-diol, hexane-1,2-diol, hexane-1,3-diol, hexane-1,4-diol, hexane-1,5-diol, hexane-1,6-diol, hexane-2,5-diol, heptane-1,2-diol, 1,7-heptanediol, 1,8-octanediol, 1,2-octanediol, 1,9-nonanediol, 1,2-decanediol, 1,10-decanediol, 1,2-dodecanediol, 1,12-dodecanediol, 1,5-hexadiene-3,4-diol, 1,2- and 1,3-cyclopentanediols, 1,2-, 1,3- and 1,4-cyclohexanediols, 1,1-, 1,2-, 1,3- and 1,4-bis-(hydroxymethyl)cyclohexanes, 1,1-, 1,2-, 1,3- and 1,4-bis(hydroxyethyl)cyclohexanes, neopentyl glycol, (2)-methyl-2,4-pentanediol, 2,4-dimethyl-2,4-pentanediol, 2-ethyl-1,3-hexanediol, 2,5-dimethyl-2,5-hexanediol, 2,2,4-trimethyl-1,3-pentanediol, pinacol, diethylene glycol, triethylene glycol, dipropylene glycol, tripropylene glycol, polyethylene glycols HO(CH₂CH₂O)ₙ—H or polypropylene glycols HO(CH[CH₃]CH₂O)ₙ—H, where n is an integer and n is ≧4, polyethylene-polypropylene glycols, where the order of the ethylene oxide or propylene oxide units may be blockwise or random, polytetramethylene glycols, preferably up to a molecular weight up to 5000 g/mol, poly-1,3-propanediols, preferably with a molecular weight up to 5000 g/mol, polycaprolactones or mixtures of two or more representatives of the above compounds. Here, one or both hydroxyl groups in the aforementioned diols can be substituted by SH groups. Preferably used diols are ethylene glycol, 1,2-propanediol, 1,3-propanediol, 1,4-butanediol, 1,5-pentanediol, 1,6-hexanediol, 1,8-octanediol, 1,2-, 1,3- and 1,4-cyclohexanediol, 1,3- and 1,4-bis(hydroxymethyl)cyclohexane, and also diethylene glycol, triethylene glycol, dipropylene glycol and tripropylene glycol.

The dihydric alcohols B₂ can optionally also comprise further functionalities, such as, for example, carbonyl, carboxyl, alkoxycarbonyl or sulfonyl, such as, for example, dimethylolpropionic acid or dimethylolbutyric acid, and also C₁-C₄-alkyl esters thereof, although the alcohols B₂ preferably have no further functionalities.

At least trifunctional alcohols (Cₓ) comprise glycerol, trimethylolmethane, trimethylolethane, trimethylolpropane, 1,2,4-butantriol, tris(hydroxymethyl)amine, tris(hydroxyethyl)amine, tris(hydroxypropyl)amine, pentaerythritol, diglycerol, triglycerol or higher condensation products of glycerol, di(trimethylolpropane), di(pentaerythritol), trishydroxymethyl isocyanurate, tris(hydroxyethyl) isocyanurate (THEIC), tris(hydroxypropyl) isocyanurate, inositols or sugars, such as, for example, glucose, fructose or sucrose, sugar alcohols, such as, for example, sorbitol, mannitol, threitol, erythritol, adonitol (ribitol), arabitol (lyxitol), xylitol, dulcitol (galactitol), maltitol, isomalt, tri- or higher-functional polyetherols based on tri- or higher-functional alcohols and ethylene oxide, propylene oxide and/or butylene oxide.

In this connection, particular preference is given to glycerol, diglycerol, triglycerol, trimethylolethane, trimethylolpropane, 1,2,4-butanetriol, pentaerythritol, tris(hydroxyethyl) isocyanurate, and polyetherols thereof based on ethylene oxide and/or propylene oxide.

The process according to the invention can be carried out without a diluent or in the presence of a solvent. Suitable solvents are, for example, hydrocarbons, such as paraffins or aromatics. Particularly suitable paraffins are n-heptane and cyclohexane. Particularly suitable aromatics are toluene, ortho-xylene, meta-xylene, para-xylene, xylene as isomer mixture, ethylbenzene, chlorobenzene and ortho- and meta-dichlorobenzene. Also suitable as solvents in the absence of acidic catalysts are very particularly ethers, such as, for example, dioxane or tetrahydrofuran and ketones, such as, for example, methyl ethyl ketone and methyl isobutyl ketone.

According to the invention, the amount of added solvent is at least 0.1% by weight, based on the mass of the starting materials to be reacted that are used, preferably at least 1% by weight and particularly preferably at least 10% by weight. It is also possible to use excesses of solvent, based on the mass of starting materials to be reacted that are used, for example 1.01 to 10-fold. Solvent amounts of more than 100-fold, based on the mass of starting materials to be reacted that are used, are not advantageous because at significantly lower concentrations of the reactants, the reaction rate diminishes significantly, which leads to uneconomical long reaction times.

In one preferred embodiment, the reaction is carried out free from solvents.

To carry out the process according to the invention it is possible to work in the presence of a water-withdrawing agent as additive, which is added at the start of the reaction. For example, molecular sieves, in particular molecular sieve 4 Å, MgSO₄ and Na₂SO₄, are suitable. During the reaction it is also possible to add further water-withdrawing agent or to replace water-withdrawing agent with fresh water-withdrawing agent. During the reaction, it is also possible to distil off formed water and/or alcohol and, for example, to use a water separator, in which the water is removed with the help of an entrainer.

Furthermore, the removal can take place by stripping, for example take place by passing a gas that is inert under the reaction conditions through the reaction mixture, if appropriate in addition to a distillation. Suitable inert gases are preferably nitrogen, noble gases, carbon dioxide or combustion gases.

The process according to the invention can be carried out in the absence of catalysts. However, preference is given to working in the presence of at least one catalyst. These are preferably acidic inorganic, organometallic or organic catalysts or mixtures of two or more acidic inorganic, organometallic or organic catalysts.

Within the context of the present invention, examples of acidic inorganic catalysts are sulfuric acid, sulfates and hydrogensulfates, such as sodium hydrogensulfate, phosphoric acid, phosphonic acid, hypophosphorous acid, aluminum sulfate hydrate, alaun, acidic silica gel (pH≦6, in particular ≦5) and acidic aluminum oxide. It is also possible to use, for example, aluminum compounds of the general formula Al(OR¹)₃ and titanates of the general formula Ti(OR¹)₄ as acidic inorganic catalysts, where the radicals R¹ may be in each case identical or different and are selected independently of one another from

C₁-C₂₀-alkyl radicals, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, isoheptyl, n-octyl, 2-ethylhexyl, n-nonyl, n-decyl, n-dodecyl, n-hexadecyl or n-octadecyl.

C₃-C₁₂-cycloalkyl radicals, for example cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl.

Preferably, the radicals R¹ in Al(OR¹)₃ and Ti(OR¹)₄ are in each case identical and selected from n-butyl, isopropyl or 2-ethylhexyl.

Preferred acidic organometallic catalysts are selected, for example, from dialkyltin oxides R¹₂SnO or dialkyltin esters R¹₂Sn(OR²)₂, where R¹ is defined as above and may be identical or different.

R² can have the same meanings as R¹ and additionally be C₃-C₁₂-aryl, for example phenyl, o-, m- or p-tolyl, xylyl or naphthyl. R² may in each case be identical or different.

Examples of organotin catalysts are tin(II) n-octanoate, tin(II) 2-ethylhexanoate, tin(II) laurate, dibutyltin oxide, diphenyltin oxide, dibutyltin dichloride, dibutyltin diacetate, dibutyltin dilaurate, dibutyltin dimaleate or dioctyltin diacetate.

Particularly preferred representatives of acidic organometallic catalysts are dibutyltin oxide, diphenyltin oxide and dibutyltin dilaurate.

Preferred acidic organic catalysts are acidic organic compounds with, for example, phosphate groups, sulfonic acid groups, sulfate groups or phosphonic acid groups. Particular preference is given to sulfonic acids, such as, for example, para-toluenesulfonic acid. It is also possible to use acidic ion exchangers as acidic organic catalysts, for example sulfonic-acid-group-containing polystyrene resins crosslinked with about 2 mol % of divinylbenzene.

It is also possible to use combinations of two or more of the aforementioned catalysts. It is also possible to use those organic or organometallic or else inorganic catalysts which are present in the form of discrete molecules in immobilized form, for example on silica gel or on zeolites.

If the desire is to use acidic inorganic, organometallic or organic catalysts, then according to the invention 0.1 to 10% by weight, preferably 0.2 to 2% by weight, of catalyst are used.

Enzymes or decomposition products of enzymes likewise belong to the organic catalysts within the context of the present invention. Particular preference is given to effective amounts of a lipase obtainable, for example, from Candida cylindracea, Candida lipolytica, Candida rugosa, Candida antarctica, Candida utilis, Chromobacterium viscosum, Geotrichum viscosum, Geotrichum candidum, Mucor javanicus, Mucor mihei, pig pancreas, Pseudomonas spp., Pseudomonas fluoprescens, Pseudomonas cepacia, Rhizopus arrhizus, Rhizopus delemar, Rhizopus niveus, Rhizopus oryzae, Aspergillus niger, Penicillium roquefortii, Penicillium camembertii or esterase from Bacillus spp. Bacillus thermoglucosidasius.

The process according to the invention is preferably carried out under an inert gas atmosphere, i.e. a gas that is inert under the reaction conditions, for example under carbon dioxide, combustion gases, nitrogen or noble gas, among which in particular argon should be mentioned.

The process according to the invention is carried out at temperatures of from 60 to 250° C. Preference is given to working at temperatures of from 80 to 200° C., particularly preferably at 100 to 180° C.

The pressure conditions of the process according to the invention are generally not critical. It is possible to work at significantly reduced pressure, for example at 10 to 500 mbar. The process according to the invention can also be carried out at pressures above 500 mbar. For reasons of simplicity, preference is given to the reaction at atmospheric pressure; however, it is also possible to carry it out at slightly increased pressure, for example up to 1200 mbar. It is also possible to work under significantly increased pressure, for example at pressures up to 10 bar. Preference is given to the reaction at reduced or atmospheric pressure, particularly preferably at atmospheric pressure.

The reaction time of the process according to the invention is usually 10 minutes to 48 hours, preferably 30 minutes to 24 hours and particularly preferably 1 to 12 hours.

When the reaction is complete, the high-functionality highly- and hyperbranched polyesters can be isolated easily, for example by filtering off the catalyst and, if appropriate, stripping off the solvent, in which case the stripping off of the solvent is usually carried out at reduced pressure. Further highly suitable processing methods are precipitation of the polymer after adding water and subsequent washing and drying.
d) If required, the reaction mixture can be subjected to a decoloring, for example through treatment with activated carbon or metal oxides, such as, for example, aluminum oxide, silicon oxide, magnesium oxide, zirconium oxide, boron oxide or mixtures thereof, in amounts of, for example, 0.1-50% by weight, preferably 0.5 to 25% by weight, particularly preferably 1-10% by weight, at temperatures of, for example, 10 to 200° C., preferably 20 to 180° C. and particularly preferably 30 to 160° C.

This can take place by adding the pulverant or granular decolorizer to the reaction mixture and subsequent filtration or by passing the reaction mixture over a bed of the decolorizer in the form of any desired suitable moldings.

The decoloring of the reaction mixture can take place at any desired point in the work-up process, for example at the stage of the crude reaction mixture or following optional prewashing, neutralization, washing or solvent removal.

The reaction mixture can furthermore be subjected to a prewashing e) and/or a neutralization f) and/or an afterwashing g), preferably merely to a neutralization f). If appropriate, the order of the neutralization f) and prewashing e) can also be swapped.

Comprised products of value can be at least partially recovered from the aqueous phase of the washing and/or neutralization by acidification and extraction with a solvent and be reused.

Prior to the pre- or afterwashing, the reaction mixture is treated in a washing apparatus with a washing liquid, for example water or a 5-30% strength by weight, preferably 5-20, particularly preferably 5-15, % strength by weight sodium chloride solution, potassium chloride solution, ammonium chloride solution, sodium sulfate solution or ammonium sulfate solution, preferably water or sodium chloride solution.

The quantitative ratio of reaction mixture:washing liquid is generally 1:0.1-1, preferably 1:0.2-0.8, particularly preferably 1:0.3-0.7.

The washing or neutralization can be carried out, for example, in a stirred container or in other conventional apparatuses, e.g. in a column or mixer-settler apparatus.

In terms of processing, all extraction and washing methods and apparatuses known per se can be used for a washing or neutralization in the process according to the invention, e.g. those described in Ullmann's Encyclopedia of Industrial Chemistry, 6th ed, 1999 Electronic Release, chapter: Liquid-Liquid Extraction-Apparatus. For example, these may be single-stage or multistage, preferably single-stage, extractions, and also those in the cocurrent or countercurrent mode, preferably countercurrent mode.

Preference is given to using sieve tray columns or columns with arranged and/or dumped packings, stirred containers or mixer-settler apparatuses, and also pulsed columns or those with rotating internals.

The prewashing is preferably used when metal salts, preferably organotin compounds, are (co)used as catalyst.

An afterwashing may be advantageous for removing traces of base or salt from the neutralized reaction mixture.

For the neutralization f), the optionally prewashed reaction mixture, which may still comprise small amounts of catalyst and/or carboxylic acid, can be neutralized with a 5-25, preferably 5-20, particularly preferably 5-15, % strength by weight aqueous solution of a base, such as, for example, alkali metal or alkaline earth metal oxides, hydroxides, carbonates or hydrogencarbonates, preferably sodium hydroxide solution, potassium hydroxide solution, sodium hydrogen carbonate, sodium carbonate, potassium hydrogen carbonate, calcium hydroxide, milk of lime, ammonia, ammoniacal water or potassium carbonate, to which, if appropriate, 5-15% by weight of sodium chloride, potassium chloride, ammonium chloride or ammonium sulfate can be added, particularly preferably with sodium hydroxide solution or sodium hydroxide solution/sodium chloride solution. The degree of neutralization is preferably 5 to 60 mol %, preferably 10 to 40 mol %, particularly preferably 20 to 30 mol %, based on the monomers comprising acid groups.

The base is added in a manner such that the temperature in the apparatus does not increase above 60° C., is preferably between 20 and 35° C. and the pH is 4-13. The heat of neutralization is preferably dissipated by means of cooling the container with the help of internal cooling coils or via jacketed cooling.

The quantitative ratio of reaction mixture:neutralization liquid is generally 1:0.1-1, preferably 1:0.2-0.8, particularly preferably 1:0.3-0.7.

As regards the apparatus, that stated above is applicable.
h) If a solvent is present in the reaction mixture, then this can be substantially removed by distillation. Preferably, any solvent present is removed from the reaction mixture after washing and/or neutralization, but, if desired, this can also take place before the washing and/or neutralization.

For this, the reaction mixture can be used with an amount of storage stabilizer such that, following removal of the solvent, 100-500, preferably 200-500 and particularly preferably 200-400 ppm thereof are present in the target ester (residue).

The distillative removal of the majority of optionally used solvent or low-boiling by-products takes place, for example, in a stirred tank with jacketed heating and/or internal heating coils under reduced pressure, for example at 20-700 mbar, preferably 30 to 500 and particularly preferably 50-150 mbar and a temperature of 40-120° C.

The distillation can of course also take place in a falling-film evaporator or thin-film evaporator. For this, the reaction mixture is conveyed through the apparatus, preferably circulated several times, under reduced pressure, for example at 20-700 mbar, preferably 30 to 500 and particularly preferably 50-150 mbar and a temperature of 40-80° C.

A gas that is inert under the reaction conditions can advantageously be introduced into the distillation apparatus, for example 0.1-1, preferably 0.2-0.8 and particularly preferably 0.3-0.7 m³ of oxygen-containing gas per m³ of reaction mixture and hour.

The residual solvent content in the residue after distillation is generally below 5% by weight, preferably 0.5-5% and particularly preferably 1 to 3% by weight.

The removed solvent is condensed and preferably reused.

If required, solvent stripping i) can be carried out in addition to or instead of the distillation.

For this, the product, which may still comprise small solvent amounts or low-boiling impurities, is heated to 50-150° C., preferably 80-150° C. and the remaining solvent amounts are removed using a suitable gas in a suitable apparatus. For assistance, if appropriate, a vacuum may also be applied.

Suitable apparatuses are, for example, columns of design known per se which have the customary internals, e.g. trays, dumped packings or ordered packings, preferably dumped packings. Suitable column internals are in principle all customary internals, for example trays, arranged packings and/or dumped packings. Of the trays, preference is given to bubble-cap trays, sieve dumped trays, valve trays, Thormann trays and/or dual-flow trays, and of the packings, preference is given to those with rings, coils, saddles, Raschig rings, Intos rings or Pall rings, barrel saddles or Interloxs saddles, Top-Pak etc., or meshes.

Also conceivable here is a falling-film evaporator, thin-film evaporator or wiped-film evaporator, such as, for example, a Luwa, Rotafilm or Sambay evaporator, which may be equipped with a demister, for example, as a splash guard.

Suitable gases are gases that are inert under the stripping conditions, in particular those which have been conditioned to a temperature of 50 to 100° C.

The amount of stripping gas is, for example, 5-20, particularly preferably 10-20 and very particularly preferably 10 to 15 m³ of stripping gas per m³ of reaction mixture and hour.

If necessary, at any desired stage of the work-up process, preferably after washing/neutralization and, if appropriate, after solvent removal, the esterification mixture can be subjected to a filtration j) in order to remove precipitated traces of salts and also any decolorizer present.

It is preferred to omit a prewash or afterwash e) or g); just a filtration step j) may be sensible. It is likewise preferred to dispense with a neutralization f).

The order of steps e)/g), and also h) and j), is arbitrary.

The present invention further provides the high-functionality, highly- or hyperbranched polyesters obtainable by the process according to the invention. They are characterized by particularly low fractions of discolorations and resinifications.

The polyesters according to the invention have a molecular weight Mₙ of at least 500, preferably at least 600 and particularly preferably 750 g/mol. The upper limit of the molecular weight Mₙ is preferably 100 000 g/mol, particularly preferably it is not more than 80 000 and very particularly preferably it is not more than 30 000 g/mol.

The data relating to the polydispersity and also to the number-average and weight-average molecular weight Mₙ and M_{w} refer here to measurements made by gel permeation chromatography using polymethyl methacrylate as standard and tetrahydrofuran, dimethylacetamide or hexafluoroisopropanol as eluent. The method is described in Analytiker Taschenbuch vol. 4, pages 433 to 442, Berlin 1984.

The polydispersity of the polyesters is 1.2 to 50, preferably 1.4 to 40, particularly preferably 1.5 to 30 and very particularly preferably up to 10.

The solubility of the polyesters is usually very good; i.e. clear solutions at 25° C. can be prepared with a content up to 50% by weight, in some cases even up to 80% by weight, of the polyesters according to the invention in tetrahydrofuran (THF), ethyl acetate, n-butyl acetate, ethanol and numerous other solvents, without gel particles being detectable by the naked eye. This demonstrates the low degree of crosslinking of the polyesters according to the invention.

The high-functionality highly- and hyperbranched polyesters are carboxy-terminated, carboxy- and hydroxy-terminated and preferably hydroxy-terminated.

In one preferred embodiment, the highly-branched polyesters are completely or partly substituted by linear or branched C₄- to C₄₀-alkyl and/or -alkenyl radicals. Within the context of the present invention, alkenyl radicals may be monounsaturated or polyunsaturated.

Within the scope of the present invention, substitution means that the highly-branched polyesters are reacted with compounds A during and/or after the polymerization reaction. Compounds A are notable for the fact that they comprise a linear or branched C₄- to C₄₀-alkyl and/or alkenyl radical and a reactive group. A reactive group of compound A is able to react with the highly-branched polyester. Preferably, compounds A comprise precisely one linear or branched C₄- to C₄₀-alkyl and/or alkenyl radical and precisely one reactive group.

Highly-branched polyesters which have been reacted with compounds A are referred to as substituted highly-branched polyesters.

The substitution can take place completely or partially. This means in the case of complete substitution that the reactive groups of the highly-branched polyester have reacted completely with compounds A. In the case of partial substitution, not all of the reactive groups of the highly-branched polyester have reacted with compounds A.

Preferably, the highly-branched polyesters are substituted by octyl (capryl), nonyl, decyl (caprinyl), undecyl, dodecyl (laurinyl), tetradecyl, hexadecyl (palmityl), heptadecyl, octadecyl (stearyl) radicals and/or the corresponding mono- or polyunsaturated equivalents, such as, for example, by dodecenyl, hexadienyl (sorbinyl), octadecenyl (oleyl), linolyl or linolenyl radicals.

In this connection, equivalent is to be understood as meaning a hydrocarbon radical which differs from the corresponding linear or branched alkyl radical only by virtue of the fact that it has at least one double bond.

The substituted highly-branched polyesters are preferably obtained by reacting the resulting high-functionality highly- or hyperbranched polyesters with a suitable functionalization reagent which can react with the OH and/or ester groups of the polyester.

High-functionality highly-branched polyesters comprising hydroxyl groups can be modified, for example, by adding acid derivative groups, such as esters, anhydrides or amides or molecules comprising isocyanate groups. For example, polyesters comprising acid groups can be obtained through reaction with compounds comprising anhydride groups.

Here, the molar ratio of the reactive groups of the substitution compound to the reactive groups of the highly-branched polyester is from 1:10 to 1:1, preferably from 1:5 to 1:1.1, especially preferably from 1:2 to 1:1.2. A particularly preferred range is 1:1.7 to 1:1.4.

In one preferred embodiment of the present invention, the substitution takes place with a carboxylic acid derivative of the formula R—CO—Y and/or an isocyanate of the formula R—NCO, where the radicals have the meaning below.

R is linear or branched C₄- to C₄₀-alkyl.

Y is OR¹, OC(O)R² or NR³₂. Here, R¹ is hydrogen or linear or branched C₁- to C₆-alkyl,

R² is linear or branched C₄- to C₄₀-alkyl, where R and R² may be identical or different.

R³ is hydrogen or linear or branched C₁- to C₄-alkyl, where the two radicals R³ may be identical or different from one another.

Preferred compounds are linear C₄-C₄₀-alkyl isocyanates, particular preference being given to octyl (capryl) isocyanate, nonyl isocyanate, decyl (caprinyl) isocyanate, undecyl isocyanate, dodecyl (laurinyl) isocyanate, tetradecyl isocyanate, hexadecyl (palmityl) isocyanate, heptadecyl isocyanate, octadecyl (stearyl) isocyanate. Further preferred compounds are linear C₄-C₄₀-alkenyl isocyanates with one or more double bonds, particular preference being given to dodecenyl, hexadienyl (sorbinyl), octadecenyl (oleyl), linolyl or linolenyl isocyanate.

A very particularly preferred compound is stearyl isocyanate.

The substitution can take place, for example, in a subsequent process step (step c)).

However, the substitution can also take place as early as during the preparation of the highly-branched polyesters.

Preferably, the substitution takes place in a subsequent process step. If the substitution takes place in a subsequent process step, then preferably the highly-branched polyester is initially introduced and one or more compounds A are added. The substitution usually takes place at a temperature from 0 to 300° C., preferably 0 to 250° C., particularly preferably at 60 to 200° C. and very particularly preferably at 60 to 160° C. without a diluent or in solution. Here, in general it is possible to use all solvents which are inert towards the particular starting materials. Preference is given to using organic solvents, such as, for example, decane, dodecane, benzene, toluene, chlorobenzene, xylene, dimethylformamide, dimethylacetamide or solvent naphtha.

In one preferred embodiment, the substitution reaction is carried out without a diluent. In order to increase the rate of the reaction, low molecular weight compounds that are released during the reaction can be removed from the reaction equilibrium, for example by distillation, if necessary under reduced pressure.

To complete the reaction, it may be necessary to raise the temperature of the reaction container following the addition of compound A or, if two or more different compounds A are used, following the addition of compounds A. The increase is usually 10 to 50° C., it is preferably 20 to 40° C.

The substitution of the high-functionality polyesters in most cases takes place in a pressure range from 0.1 mbar to 20 bar, preferably at 1 mbar to 5 bar, in reactors or reactor cascades which are operated in batch operation, semicontinuously or continuously.

The invention provides a cosmetic composition comprising at least one substituted highly-branched polyester.

The cosmetic composition preferably comprises at least one cosmetically suitable carrier.

The use of a substituted highly-branched polyester in cosmetic and/or dermatological formulations is in accordance with the invention.

Preference is given to the use in skin cosmetic formulations.

Preference is given to using a substituted highly-branched polyester as thickener. In this connection, in particular the use as oil thickener is preferred.

### Skin Cosmetic Preparations

Skin cosmetic compositions according to the invention, in particular those for skincare, may be present and used in various forms. Thus, for example, they may be an emulsion of the oil-in-water (O/W) type or a multiple emulsion, for example of the water-in-oil-in-water (W/O/W) type. Emulsifier-free formulations such as hydrodispersions, hydrogels or a Pickering emulsion are also advantageous embodiments.

The consistency of the formulations can range from pasty formulations via flowable formulations to low viscosity, sprayable products. Accordingly, creams, lotions or sprays can be formulated. For use, the cosmetic compositions according to the invention are applied in an adequate amount to the skin in the manner customary for cosmetics and dermatological compositions.

The salt content in the surface of the skin is sufficient to lower the viscosity of the preparations according to the invention in such a way as to facilitate simple spreading and working-in of the preparations.

The skin cosmetic preparations according to the invention are present in particular as W/O or O/W skin creams, day and night creams, eye creams, face creams, antiwrinkle creams, mimic creams, moisturizing creams, bleaching creams, vitamin creams, skin lotions, care lotions and moisturizing lotions.

Further advantageous skin cosmetic preparations are face toners, face masks, deodorants and other cosmetic lotions and preparations for decorative cosmetics, for example concealing sticks, stage make-up, mascara, eyeshadows, lipsticks, kohl pencils, eyeliners, make-ups, foundations, blushers, powders and eyebrow pencils. Moreover, the compositions according to the invention can be used in nose strips for pore cleansing, in antiacne compositions, repellants, shaving compositions, hair removal compositions, intimate care compositions, foot care compositions, and in baby care. Besides the W/W emulsion polymer and suitable carriers, the skin cosmetic preparations according to the invention also comprise further active ingredients and/or auxiliaries customary in cosmetics, as described above and below.

These include preferably emulsifiers, preservatives, perfume oils, cosmetic active ingredients, such as phytantriol, vitamin A, E and C, retinol, bisabolol, panthenol, natural and synthetic photoprotective agents, bleaches, colorants, tinting agents, tanning agents, collagen, protein hydrolyzates, stabilizers, pH regulators, dyes, salts, thickeners, gel formers, consistency regulators, silicones, humectants, conditioners, refatting agents and further customary additives.

Further polymers may also be added to the compositions if specific properties are to be set. To establish certain properties, such as, for example, improving the feel to the touch, the spreading behavior, the water resistance and/or the binding of active ingredients and auxiliaries such as pigments, the compositions can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

Further possible ingredients of the compositions according to the invention are described below under the respective keyword.

### Oils, Fats and Waxes

The skin and hair cosmetic compositions preferably also comprise oils, fats or waxes. Constituents of the oil phase and/or fatty phase of the cosmetic compositions are advantageously selected from the group of lecithins and fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be advantageously selected from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be selected from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and also from the group of esters of aromatic carboxylic acids and saturated and/or unsaturated, branched and/or unbranched alcohols of chain length from 3 to 30 carbon atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and also synthetic, semisynthetic and natural mixtures of such esters, such as, for example, jojoba oil.

In addition, one or more oil components can be advantageously selected from the group of branched and unbranched hydrocarbons and hydrocarbon waxes, the silicone oils, the dialkyl ethers, the group of saturated or unsaturated, branched or unbranched alcohols.

Any desired mixtures of such oil and wax components are also to be used advantageously within the context of the present invention. It may in some instances also be advantageous to use waxes, for example cetyl palmitate, as the sole lipid component of the oil phase.

According to the invention, the oil component is advantageously selected from the group consisting of 2-ethylhexyl isostearate, octyldodecanol, isotridecyl isononanoate, isoeicosane, 2-ethylhexyl cocoate, C12-15-alkyl benzoate, caprylic-capric triglyceride, dicaprylyl ether.

Mixtures of C12-C15-alkyl benzoate and 2-ethylhexyl isostearate, mixtures of C12-C15-alkyl benzoate and isotridecyl isononanoate, and also mixtures of C12-C15-alkyl benzoate, 2-ethylhexyl isostearate and isotridecyl isononanoate are advantageous according to the invention.

According to the invention, as oils with a polarity of from 5 to 50 mN/m, particular preference is given to using fatty acid triglycerides, in particular soybean oil and/or almond oil.

Of the hydrocarbons, paraffin oil, squalane, squalene and in particular polyisobutenes, which may also be hydrogenated, are to be used advantageously within the context of the present invention.

In addition, the oil phase can be advantageously selected from the group of Guerbet alcohols. Guerbet alcohols are produced by the reaction equation

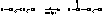

by oxidation of an alcohol to give an aldehyde, by aldol condensation of the aldehyde, elimination of water from the aldol and hydrogenation of the allylaldehyde. Guerbet alcohols are liquid even at low temperatures and cause virtually no skin irritations. They can be used advantageously as fatting, superfatting and also refatting constituents in cosmetic compositions.

The use of Guerbet alcohols in cosmetics is known per se. Such species are then characterized in most cases by the structure

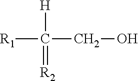

Here, R¹ and R² are generally unbranched alkyl radicals.

According to the invention, the Guerbet alcohol or alcohols are advantageously selected from the group where

R₁=propyl, butyl, pentyl, hexyl, heptyl or octyl and
R₂=hexyl, heptyl, octyl, nonyl, decyl, undecyl, dodecyl, tridecyl or tetradecyl.

Guerbet alcohols preferred according to the invention are 2-butyloctanol (commercially available, for example, as Isofol® 12 (Condea)) and 2-hexyldecanol (commercially available, for example, as Isofol® 16 (Condea)).

Mixtures of Guerbet alcohols according to the invention are also to be used advantageously according to the invention, such as, for example, mixtures of 2-butyl-octanol and 2-hexyldecanol (commercially available, for example, as Isofol® 14 (Condea)).

Any desired mixtures of such oil and wax components are also to be used advantageously within the context of the present invention. Among the polyolefins, polydecenes are the preferred substances.

The oil component can advantageously also have a content of cyclic or linear silicone oils or consist entirely of such oils, although it is preferred to use an additional content of other oil phase components besides the silicone oil or the silicone oils.

Low molecular weight silicones or silicone oils are generally defined by the following general formula

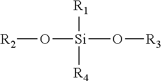

Higher molecular weight silicones or silicone oils are generally defined by the following general formula

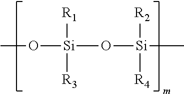

where the silicon atoms can be substituted by identical or different alkyl radicals and/or aryl radicals, which are shown here in general terms by the radicals R₁ to R₄. However, the number of different radicals is not necessarily limited to 4. m can here assume values from 2 to 200 000.

Cyclic silicones to be used advantageously according to the invention are generally defined by the following general formula

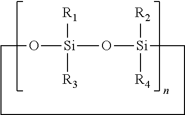

where the silicon atoms can be substituted by identical or different alkyl radicals and/or aryl radicals, which are represented here in general terms by the radicals R₁ to R₄. However, the number of different radicals is not necessarily limited to 4. n can here assume values from 3/2 to 20. Fractional values for n take into consideration that odd numbers of siloxyl groups may be present in the cycle.

Phenyltrimethicone is advantageously selected as silicone oil. Other silicone oils, for example dimethicone, hexamethylcyclotrisiloxane, phenyldimethicone, cyclomethicone (e.g. decamethylcyclopentasiloxane), hexamethylcyclotrisiloxane, polydimethylsiloxane, poly(methylphenylsiloxane), cetyldimethicone, behenoxydimethicone are also to be used advantageously within the context of the present invention. Also advantageous are mixtures of cyclomethicone and isotridecyl isononanoate, and also those of cyclomethicone and 2-ethylhexyl isostearate. However, it is also advantageous to select silicone oils of similar constitution to the compounds referred to above, the organic side chains of which are derivatized, for example polyethoxylated and/or polypropoxylated. These include, for example, polysiloxanepolyalkyl-polyether copolymers, such as, for example, cetyl-dimethicone copolyol.

Cyclomethicone (octamethylcyclotetrasiloxane) is advantageously used as silicone oil to be used according to the invention.

Fat and/or wax components to be used advantageously can be selected from the group of vegetable waxes, animal waxes, mineral waxes and petrochemical waxes. For example, candelilla wax, carnauba wax, Japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and micro waxes. Further advantageous fat and/or wax components are chemically modified waxes and synthetic waxes, such as, for example, Syncrowax®HRC (glyceryl tribehenate), and Syncrowax®AW 1 C (C₁₈₋₃₆-fatty acid), and also montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example Tegosoft®CR, polyalkylene waxes, polyethylene glycol waxes, but also chemically modified fats, such as, for example, hydrogenated plant oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Also certain organosilicon compounds which have similar physical properties to the specified fat and/or wax components, such as, for example, stearoxytrimethylsilane, are furthermore advantageous.

According to the invention, the fat and/or wax components can be used either individually or as a mixture in the compositions.

Any desired mixtures of such oil and wax components are also to be used advantageously within the context of the present invention.

The oil phase is advantageously selected from the group consisting of 2-ethylhexyl isostearate, octyldodecanol, isotridecyl isononanoate, butylene glycol dicaprylate/dicaprate, 2-ethylhexyl cocoate, C12-15-alkyl benzoate, caprylic-capric triglyceride, dicaprylyl ether.

Mixtures of octyldodecanol, caprylic-capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and also mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously within the context of the present invention.

The oil component can also be advantageously selected from the group of phospholipids. The phospholipids are phosphoric acid esters of acylated glycerols. Of greatest importance among the phosphatidylcholines are, for example, the lecithins, which are characterized by the general structure

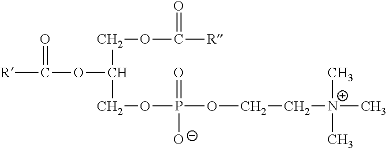

where R′ and R″ are typically unbranched aliphatic radicals having 15 or 17 carbon atoms and up to 4 cis double bonds.

According to the invention, as paraffin oil advantageous according to the invention it is possible to use Merkur Weissoel Pharma 40 from Merkur Vaseline, Shell Ondina® 917, Shell Ondina® 927, Shell Oil 4222, Shell Ondina° 933 from Shell & DEA Oil, Pionier® 6301 S, Pionier® 2071 (Hansen & Rosenthal).

Suitable cosmetically compatible oil and fat components are described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is hereby made in its entirety.

Further embodiments of the present invention are given in the claims, the description and the examples. It goes without saying that the features of the subject matter according to the invention that have been specified above and are still to be explained below can be used not only in the combination stated in each case, but also in other combinations, without departing from the scope of the invention.

The present invention will be illustrated by the examples below.

### EXAMPLES

### Measurement Methods

The IR measurements were carried out using a Nicolet 210 instrument.

The acid number and the hydroxyl number were determined in accordance with DIN 53240, part 2.

The molecular weight was determined with the help of gel permeation chromatography using a refractometer as detector. The mobile phase used was dimethylacetamide, and the standard used for determining the molecular weight was polymethyl methacrylate (PMMA).

### Feed Materials

DBTL: Dibutyltin dilaurate, manufacturer: Sigma-Aldrich

### Paraffin oil: Nujol, Fluka AG

### Example 1

### Preparation of a Highly-Branched Polyester

233.2 g of adipic acid (1.6 mol) and 266.0 g (1.33 mol) of a triol based on trimethylolpropane which has been etherified in a random manner with 1,2-propylene oxide units were initially introduced into a 1000 ml glass reactor fitted with stirrer, reflux condenser, gas inlet, attached thereto a vacuum system with interconnected cold trap and internal thermometer. After adding 200 ppm of DBTL, the mixture was heated to 150° C. During this, the internal pressure was reduced to a final value of ca. 10 mbar in such a way that the water that formed was removed in a controlled manner. Stirring was carried out for 5.5 hours at this temperature. The acid number was 68 mg KOH/g. 97 g of the aforementioned triol (0.8 equivalents per acid group) were added to the reaction mixture. The reaction mixture was stirred for a further 5 hours at an internal pressure of ca. 10 mbar and then cooled to ambient temperature at this pressure. The end product was obtained as viscous, clear liquid which has the following properties: acid number=26 mg KOH/g; hydroxyl number=204 mg KOH/g; viscosity: 6800 mPas (75° C.)

### Examples 2-8

### Modification of the Highly-Branched Polyester with Stearyl Isocyanate

Highly-branched polyester from example 1 was initially introduced into a 250 ml glass reactor equipped with stirrer, reflux condenser, gas inlet, internal thermometer and dropping funnel, which comprised the required amount of stearyl isocyanate. The amounts of highly-branched polyester and stearyl isocyanate used are given in the table below.

The reactor was heated to 80° C. and the isocyanate was added dropwise over the course of 15 minutes. The reaction mixture was then stirred for a further 2 hours at 120° C. and the reaction progress was monitored via the disappearance of the isocyanate groups with the help of IR spectroscopy (disappearance of the isocyanate band at 2270 cm⁻¹).

| | Example | | | | | | |
|---|---|---|---|---|---|---|---|
| | 2 | 3 | 4 | 5 | 6 | 7 | 8 |
| Highly-branched | 50 | 50 | 51.2 | 76.6 | 50 | 50 | 50 |
| polyester (example 1) | | | | | | | |
| [g] | | | | | | | |
| Mol % NCO | 40 | 50 | 60 | 70 | 80 | 90 | 100 |
| Amount of stearyl | 21.5 | 26.9 | 33.1 | 58 | 43.04 | 48.4 | 53.8 |
| isocyanate [g] | | | | | | | |

### Example 9

### Gel Formation by Adding the Stearyl-Modified Highly-Branched Polyester to Paraffin Oil

Various amounts (0.5 to 20% by weight) of the polymers in examples 3 to 8 were dissolved in paraffin oil. The concentration at which visible gel formation occurred is given in the table below:

| | Polymer from example . . . | | | | | |
|---|---|---|---|---|---|---|
| | 3 | 4 | 5 | 6 | 7 | 8 |
| NCO/OH ratio [%] | 50 | 60 | 70 | 80 | 90 | 100 |
| Gel formation | 10 | 10 | 7 | 6 | 4 | 4 |
| concentration (%) | | | | | | |

1.-8. (canceled)

9. A highly branched polyester
which is completely or partly substituted by linear or branched C₄- to C₄₀-alkyl- or alkenyl radicals.

10. The substituted highly branched polyester according to claim 9,
wherein the substitution takes place with a derivative of the formula R—CO—Y and/or R—NCO
where
R=linear or branched C₄- to C₄₀-alkyl,
Y=OR′, OC(O)R², NR³₂ or halogen
R¹=hydrogen, linear or branched C₁- to C₆-alkyl,
R²=linear or branched C₄- to C₄₀-alkyl, where R and R² may be identical or different,
R³=hydrogen, linear or branched C₁- to C₄-alkyl, where the two radicals R³ may be identical or different from one another.

11. A cosmetic composition comprising at least one highly branched polyester according to claim 9.

12. The cosmetic composition according to claim 11, comprising at least one cosmetically suitable carrier.

13. A cosmetic or dermatological formulation which comprises the highly branched polyester according to claim 9.

14. A skin cosmetic formulation which comprises the highly branched polyester according to claim 9.

15. A thickener which comprises the highly branched polyester according to claim 9.

16. An oil thickener which comprises the highly branched polyester according to claim 9.
